(19) United States
(12) Patent Application Publication
(10) Pub. No.: US 2025/0344963 A1
(43) Pub. Date: Nov. 13, 2025

(54) METHODS FOR ACQUIRING AND ANALYZING NEUROMELANIN-SENSITIVE MRI

(71) Applicants: UNIVERSITY OF OTTAWA INSTITUTE OF MENTAL HEALTH RESEARCH, Ottawa (CA); Terran Biosciences Inc., Miami Beach, FL (US)

(72) Inventors: Clifford CASSIDY, Ottawa (CA); Samuel CLARK, Miami Beach, FL (US)

(21) Appl. No.: 18/869,507

(22) PCT Filed: May 31, 2023
(86) PCT No.: PCT/US2023/067713

§ 371 Date: Nov. 26, 2024

Related U.S. Application Data
(60) Provisional application No. 63/347,266, filed on May 31, 2022.
(60) Provisional application No. 63/348,338, filed on Jun. 2, 2022.

(51) Int. Cl. A61B 5/055; A61B 5/00; G16H 30/40; G16H 50/20

(52) U.S. Cl. CPC A61B 5/055; A61B 5/0042; A61B 5/4082; A61B 5/4088; A61B 5/742; G16H 30/40; G16H 50/20

(57) ABSTRACT

A neuromelanin sensitive magnetic resonance imaging (“MRI”) technique, method and computer-accessible medium for measuring the extent of, providing a diagnosis of, monitoring the treatment of, assessing novel treatments for, or determining a prognosis related to one or more neurological conditions. To support these applications, the present disclosure accurately determines the normative range of neuromelanin-sensitive MRI signal and volume metrics in cognitively normal older adults. Those displaying certain characteristic neuromelanin-sensitive MRI signals falling outside of the normative range should be assessed and treated according to the particular diagnosis as provided by the present application.

### CROSS REFERENCE TO RELATED APPLICATIONS

The present application claims priority to and the benefit of U.S. Provisional Patent Application Nos. 63/347,266 and 63/348,338, filed May 31, 2022, and Jun. 2, 2022, respectively, the disclosures of each of which are incorporated by reference herein for all purposes.

The present application relates to U.S. application Ser. Nos. 17/226,375, 17/636,018, 18/171,276, and PCT Application Nos. PCT/US2021/059590, PCT/US2022/020036, the disclosures of each of which are incorporated by reference herein for all purposes.

### FIELD OF THE DISCLOSURE

The present disclosure relates generally to magnetic resonance imaging (“MRI”), and more specifically, to exemplary embodiments of an exemplary system, method and computer-accessible medium for a neuromelanin-sensitive MRI technique as a non-invasive measure of neurological conditions.

### BACKGROUND

Neurological disorders diseases can be divided into two all-encompassing wide categories of brain afflictions: 1. Conditions affecting memory that are ordinarily related to dementia such as Alzheimer's disease and 2. Conditions causing problems with movements such as Parkinson's. The most widely known neurological disorders include Alzheimer (or Alzheimer's) disease along with its precursor mild cognitive impairment (MCI), Parkinson's disease (including Parkinson's disease dementia), and multiple sclerosis and a host of others. Less well-known neurological disorders include dozens of names in a comprehensive listing found at the web site of the National Institute of Neurological Disorders and Stroke (NINDS) of the National Institutes of Health (NIH) of the United States.

For many neurological disorders there are absolute diagnoses to date and there is a great clinical need for developing sensitive non-invasive diagnostics for treating these conditions. Diagnosis and monitoring of subjects with neurological disorders is critical for assessing severity of progression to respond with the appropriate preventative care. During the onset disease, timely intervention could be life-saving. A comprehensive imaging modality for assessing Alzheimer's disease remains a significant unmet clinical need.

Differentiating between different disorders with similar clinical presentations solely based on presenting symptoms is difficult because the symptoms often overlap between related conditions. There are currently no FDA cleared software as medical devices for the measurement of neuromelanin in the SN or LC which can provide a treatment regimen for one or more neurological disorders. There is a need for an improved ability to differentiate between related disorders, which can drive improved clinical outcomes.

### SUMMARY

In one aspect, the present disclosure provides a system comprising:
an MRI system configured to generate and acquire a neuromelanin-sensitive MRI scan along with a neuromelanin data series for a voxel or segment located within a region of interest in a subject's brain;
a signal processor configured to process the series of neuromelanin data to produce a processed neuromelanin MRI spectrum; and
a diagnostic processor configured to process the signal processed neuromelanin MRI spectrum, and
   extract a level, signal and/or concentration of neuromelanin in the left SN, right SN, left LC, and right LC;
   compare the measurement to a pre-determined control level, signal and/or concentration of neuromelanin in the left SN, right SN, left LC, and right LC,
   identifying a neurological disorder if the level, signal and/or concentration of neuromelanin in the left SN, right SN, left LC, and right LC deviates from the control by a predetermined amount.

In one aspect, the present disclosure provides a method of treating a neurological disorder comprising:
(i) obtaining a neuromelanin-sensitive Magnetic Resonance Imaging (MRI) scan;
(ii) determining the level, signal and/or concentration of neuromelanin in the left SN, right SN, left LC, and right LC;
(ii) comparing the level, signal and/or concentration of neuromelanin in the left SN, right SN, left LC, and right LC to a pre-determined control level, signal and/or concentration of neuromelanin in the left SN, right SN, left LC, and right LC;
(iii) administering a treatment to the subject if the level, signal and/or concentration of neuromelanin in one or more of the left SN, right SN, left LC, and right LC deviates from the control by a predetermined amount.

In one aspect, the present disclosure provides a method for treating a neurological disorder in a subject in need thereof comprising:
a) performing a neuromelanin-sensitive MRI scan on the subject;
b) acquiring a level, signal and/or concentration of neuromelanin in the left SN, right SN, left LC, and right LC;
c) comparing the level, signal and/or concentration of neuromelanin in the left SN, right SN, left LC, and right LC in a region of interest in the subject's brain with a predetermined control from each region;
d) providing an identification of the neurological disorder based on the comparing of (c);
e) administering to a subject a therapeutic for the treatment of the identified neurological disorder;
f) performing one or more subsequent neuromelanin-sensitive MRI scans on the subject.

In one aspect, the present disclosure provides a method for distinguishing between neurological disorders with similarly presenting symptoms comprising:
(i) performing an examination;
(ii) obtaining a neuromelanin-sensitive Magnetic Resonance Imaging scan at a first time point;
(iv) performing a voxel-based analysis and determining a concentration and/or volume of neuromelanin in the SNc;
(v) performing a segmented based analysis and determining a concentration and/or volume of neuromelanin in the LC;
(vi) comparing the level, concentration and/or volume of neuromelanin in the SNc to a standard value;
(vii) comparing the level, concentration and/or volume of neuromelanin in the LC to a standard value;
(viii) distinguishing between the neurological disorders based on the deviation of the level, concentration and/or volume of neuromelanin SNc and/or LC and a control.

In one aspect, the present disclosure provides a method of diagnosing a subject with a neurological disorder, said method comprising:
(i) measuring a concentration and/or volume of neuromelanin in the SNc using a voxel-based analysis method and measuring a concentration and/or volume of neuromelanin in the LC using a segmented based analysis method;
(ii) comparing the level of neuromelanin in the SNc to a standard control level of neuromelanin in the SNc and comparing the level of neuromelanin in the LC to a standard control level of neuromelanin in the LC,
(iii) providing a diagnosis of the neurological condition if the magnitude or ratio of SNc and LC neuromelanin is lower or higher for each of their respective regions relative to the control.

In one aspect, the present disclosure provides a method of determining if a subject has or is at risk of developing a neurological disorder, the method comprising analyzing one or more neuromelanin-sensitive Magnetic Resonance Imaging scans of the subject's brain region of interest, wherein the analyzing comprises:
receiving imaging information of the brain region of interest; and
determining a neuromelanin level or concentration in the brain region of interest using segmented analysis based on the imaging information;
   wherein the determining if a subject has or is at risk of developing the neurological disorder comprises:
(1) if the one or more neuromelanin-sensitive MRI scans has a decreased neuromelanin signal compared to a one or more control level without a neurological disorder then the subject has or is at risk of developing the neurological disorder; or
(2) if the one or more neuromelanin-sensitive MRI scans have a neuromelanin signal within one standard deviation of one or more control level then the subject does not have or is not at risk of developing the neurological disorder.

In one aspect, the present disclosure provides a method for distinguishing between neurological disorders with similarly presenting symptoms comprising:
(i) performing an examination to determine a Unified Parkinson's Disease Rating Scale score;
(ii) obtaining a neuromelanin-sensitive MRI scan;
(iv) performing a voxel-based analysis and determining a level, concentration and/or volume of neuromelanin in the SNc;
(v) performing a segmented based analysis and determining a level, concentration and/or volume of neuromelanin in the LC;
(vi) comparing the level, concentration and/or volume of neuromelanin in the SNc to a standard value;
(vii) comparing the level, concentration and/or volume of neuromelanin in the LC to a standard value;
(viii) identifying the neurological disorder.

### BRIEF DESCRIPTION OF THE DRAWINGS

FIG. 1 shows an SN map illustrating voxelwise values for baseline SN signal.

FIG. 2 shows the values of all baseline summary metrics for all participants.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

### Definitions

The particulars shown herein are by way of example and for purposes of illustrative discussion of the embodiments of the present disclosure only and are presented in the cause of providing what is believed to be the most useful and readily understood description of the principles and conceptual aspects of the present disclosure. In this regard, no attempt is made to show structural details of the present disclosure in more detail than is necessary for the fundamental understanding of the present disclosure, the description is taken with the drawings making apparent to those skilled in the art how the forms of the present disclosure may be embodied in practice.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not to be considered as an attempt to limit the application of the doctrine of equivalents to the scope of the claims, each numerical parameter should be construed in light of the number of significant digits and ordinary rounding conventions.

Additionally, the disclosure of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range. Moreover, the terminology at least includes the stated number, e.g., “at least 50” includes 50.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within +10% of the recited value. For example, in some instances, “about 100 [units]” may mean within +10% of 100 (e.g., from 90 to 110). The terms “about” and “approximately” may be used interchangeably.

The term “MR” refers to magnetic resonance and is the physical principle upon which a variety of experimental procedures known in the art and/or described herein are based, including MRI (“magnetic resonance imaging”), MRS (“magnetic resonance spectroscopy”) and the like. The term “neuromelanin-sensitive MRI” or “neuromelanin-MRI” refer to the use of MRI in the evaluation of neuromelanin in the brain. Herein the general term magnetic resonance image, magnetic resonance imaging or MRI encompasses neuromelanin-sensitive variants.

As used herein, the term “NM-MRI” and similar nomenclature refers to each the MRI scan and corresponding voxel wise analysis independently, both as separate and together.

The terms “T1” and “T2” used herein refer to the conventional meanings well known in the art (i.e., “spin-lattice relaxation time,” and “spin-spin relaxation time,” respectively).

The term “T1-weighted” in the context of MRI images refers to an image made with pulse spin echo or inversion recovery sequence, having appropriately shortened TR and TE, which as known in the art can demonstrate contrast between tissues having different T1 values. The term “TR” in this context refers to the repetition time between excitation pulses. The term “excitation pulse” is understood to refer to a 90-deg radio frequency (RF) excitation pulse. The term “TE” refers to the echo time between the excitation pulse and MR signal sampling.

The term “subject” may be a mammalian subjects such as murine, rattus, equine, bovine, ovine, canine, feline or human. In some embodiments of the methods described herein, the subject is a mouse, while in other embodiments the subject is a human. The term “subject” in this context refers to a human subject.

As used herein, the term “alleviate” is meant to describe a process by which the severity of a sign or symptom of a disorder is decreased. Importantly, a sign or symptom can be alleviated without being eliminated. In a preferred embodiment, the use of treatment methods disclosed herein leads to the elimination or reduction of a sign or symptom, however, elimination is not required. Effective dosages guided by the present disclosure are expected to decrease the severity of a sign or symptom.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug interaction(s), reaction sensitivities, and tolerance/response to therapy. In some embodiments, an effective amount of a pharmaceutical agent can be that which provides an objectively identifiable improvement.

The term “neurological condition” is used interchangeably with “neurological disorder” and “neurological disease” and is intended to encompass the conditions/disorders known in the art, at least several of which have been enumerated herein, including “neurodegenerative disorders”.

As used herein, “stable” refers to measurements that are reproducible. In one embodiment, “stable neuromelanin levels” refers to serial scans where neuromelanin levels remain approximately constant. In some contexts, “stable neuromelanin levels” are maintained for one or more hours, one or more days, one or more weeks, or one or more treatment cycles.

The terms “treat,” “treatment” and/or the like, in the context of disease, refer to ameliorating, suppressing, eradicating, and/or delaying the onset of the disease being treated. In some embodiments, the methods described herein are conducted with subjects in need of treatment. The terms “in need of treatment” and the like as used herein refer to a subject at risk for developing a disease, having a condition, which would be understood by those of skill in the medical or veterinary arts as likely leading to a disease, and/or actually having a disease. For example, Alzheimer's disease treatments include currently approved and investigative treatments. Conventional MRI lacks the spatial and quantitative data needed to predict clinical outcomes. However, the methods as discussed herein detect levels of neuromelanin in the brain that can predict clinical progression, severity, and response in Alzheimer's disease given the variance of neuromelanin in the brain or loss of neuromelanin-containing neurons.

In some embodiments, neuromelanin-sensitive MRI technique disclosed herein measures neuromelanin directly or indirectly. In some embodiments, the technique measures dopamine function directly or indirectly. In some embodiments, there is a connection between neuromelanin-sensitive MRI (NM-MRI) signal and Alzheimer's disease severity.

In some embodiments, the neuromelanin-sensitive MRI technique is capable of determining the concentrations of neuromelanin across all sections of brain tissue.

In other embodiments, the neuromelanin-sensitive MRI technique is capable of determining regional concentrations of neuromelanin.

In other embodiments, the neuromelanin-sensitive MRI technique is capable of determining regional levels of neuromelanin.

In other embodiments, the neuromelanin-sensitive MRI technique is capable of determining regional signal intensity of neuromelanin.

In other embodiments, the neuromelanin-sensitive MRI technique determines the neuromelanin concentration in the locus coeruleus (LC) subregions.

In further embodiments, the neuromelanin-sensitive MRI technique determines dopamine release in the dorsal striatum and resting blood flow within the locus coeruleus either directly or indirectly.

In some embodiments, one or more measurements in a subject of each of symptom-specific segments, or disease-specific segments, or neuromelanin concentrations, or neuromelanin volumes of specific regions or subregions may be combined with information from a second imaging test including PET imaging, fMRI, and BOLD (e.g., forming a combination of two readings or two measurements) to more accurately identify and treat the neurological disorder.

In some embodiments, the level of neuromelanin in the SNc is measured with the voxel-based algorithm and the level of neuromelanin in the LC is measured via the segmented based algorithm.

In some embodiments, these two readings together enable an accurate disease identification compared to using either reading alone.

In some embodiments, the disease identification is followed by administration of a treatment corresponding with the disease identification.

In some embodiments the concentration, volume, signal, and/or level of neuromelanin in the SNc is measured with a voxel-based algorithm.

In some embodiments the concentration, volume, signal, and/or level of neuromelanin in the LC is measured via a segmented based algorithm.

In some embodiments, the concentration, volume, signal, and/or level of neuromelanin in the SNc is measured with the voxel-based algorithm and the level of neuromelanin in the LC is measured via the segmented based algorithm.

In some embodiments, combining the two measurements enables a more precise diagnosis than using either measurement algorithm alone. In some embodiments, combining the two measurements enables distinguishing between diagnoses compared to using either measurement algorithm alone. In some embodiments, combining the two measurements enables distinguishing between similar diagnoses and selecting a more useful treatment regimen compared to using either measurement algorithm alone. In some embodiments, the control signal and/or volume in each of the left SN, right SN, left LC, and right LC are shown in the Table A below.

TABLE A
Disorder Identification and Treatment Selection Based
on Deviation from Normal Neuromelanin Levels

| | | SN | | | LC | | |
|---|---|---|---|---|---|---|---|
| | | | | T stat | | | T stat |
| | | | Annual | (annual | | Annual | (annual |
| | | Baseline | change | change) | Baseline | change | change) |
| Signal | Left | 10.02 ± 1.48 | −0.39 ± 1.32 | −1.89 | 24.71 ± 4.88 | 0.67 ± 3.29 | 1.28 |
| (CNR) | Right | 10.28 ± 1.51 | −0.30 ± 1.18 | −1.61 | 20.42 ± 4.95 | −0.62 ± 2.92 | −1.35 |
| Volume | Left | 576.4 ± 83.0 | −30.44 ± 75.9 | −2.57* | 6.31 ± 1.22 | 0.27 ± 0.92 | 1.83 |
| (mm³) | Right | 540 ± 74.9 | −17.21 ± 64.2 | −1.72 | 6.30 ± 1.20 | 0.30 ± 1.37 | 1.41 |

*p = 0.014

### LC and SNc Dual Analysis

In one embodiment, a neurological disorder can be determined using any of the methods discussed herein according to the following table:

| | SNc neuromelanin | |
|---|---|---|
| | CNR level | LC neuromelanin CNR level |
| Neurological | determined with the | determined with the |
| Condition/Indication | voxel based algorithm | segmented based algorithm |
| Schizophrenia with | Greater than one | Normal/Unchanged |
| psychotic symptoms | standard deviation | |
| (prognostic) | above 10.28 | |
| Cocaine use disorder | About 14% greater | Trend to lower than 20 |
| (diagnostic) | than 10.28; range about | |
| | 10% to about 20% | |
| Parkinson's disease | Low a level <8 or | Normal/Unchanged |
| (diagnostic) | about 23% lower than | |
| | 10.28 or a change | |
| | (reduction) in NM of | |
| | about 10% | |
| Alzheimer's disease without | Within one standard | 10% or more less than 20 |
| neuropsychiatric symptoms | deviation of 10 | |
| (diagnostic) | | |
| Alzheimer's disease | Normal or Less than 10 | Above 24 |
| neuropsychiatric symptoms | | |
| controlling for the severity | | |
| of the illness (prognostic) | | |
| Major depressive disorder | Within one standard | 10% or more less than 20 |
| (prognostic) | deviation of 10 | |
| PTSD (diagnostic and | Within one standard | Above 24 |
| prognostic) | deviation of 10 | Range greater than |
| | | Also the higher the |
| | | neuromelanin level the more |
| | | severe the symptoms |

In some embodiments, according to any of the methods described herein, the changes detected between neuromelanin-sensitive MIRI scans or against a standard control are used to provide a prognosis a neurological condition according to the Table above.

### Treatments Based on Disorder Identification

Depending on the disorder identification by the methods and systems of the present disclosure, one or more treatments are selected for administration to a subject in need thereof.

These treatments may include stellate ganglion block, vagus nerve stimulation, venlafaxine, beta blocker, prazosin, brexpiprazole and aripiprazole, iloperidone, and 3,4-Methylenedioxymethamphetamine (MDMA), selective serotonin reuptake inhibitors (SSRIs), SNRIs, NMDA antagonists including ketamine.

Exemplary Alzheimer's treatments: a cholinesterase inhibitor and/or a NMDA receptor antagonist. Exemplary and nonlimiting agents for treating Alzheimer's disease that may be administered include Leqembi (lecanemab-irmb), Donepezil (Aricept), Galantamine (Razadyne), Rivastigmine (Exelon), Memantine (Namenda), Memantine (Namzaric), Ginkgo biloba extract, Aducanumab, 3APS, 3TC, AAB-001, AADvacl, AAV2-BDNF gene therapy, ABBV-8E12, ABBV-552, ABBV-916, ABT-089, ABT-126, ABT-288, ABT-384, ABvac40, AC-1204, AC-3933, AC-OLE-01-VA, ACC-001, acetyl-L-carnitine, ACI-24.060, ACI-35.030, acitretin, active tPBM, ACU193, acumor XL, AD-35, adenosine triphosphate, adlarity, aducanumab, aduhelm, AGB101, akpha E, AL001, AL002, albumin 5%, albumin 20%, alfoatirin, allogeneic MSC, allopregnanolone, ALN-APP, ALZ-101, alzhok, ALZT-OP1a, ALZT-OP1b, amlodipine, AMX0035, AN-1792, ANAVEX2-73, APH-1105, APNmAb005, Aqua Gem-E, Aquasol E, AQW051, AR1001, ARBs, aricept, aricept ODT, aripiprazole, armodafinil, astaxanthin, AstroStem, atabecestat, ATH-1017, atomoxetine hydrochloride, atorvastatin, atorvastatin calcium, atropine, AV-1959D, avagacestat, AVE1625, AVP-786, AVP-923-30, AXS-05 (dextromethorphan-bupropion), AZD0530, AZD1446, AZD3480, AZD3480, AZD5213, azeliragon, BAC, bapineuzumab, baricitinib, belsomra, benfotiamine, bepranemab, bexarotene, BI 409306, BI 425809, bifeprunox, BIIB080, BIIB092, BMS-708163, BPDO-1603, BPN14770, brexpiprazole, bromocriptine, bryostatin, bryostatin 1, buntanetap, buprenorphine, C-11 ER-176, CAD106 Immunotherapy, canakinumab, caprylic triglyceride, caprylidene, carvedilol, CB-AC-02, CCB, celecoxib (Celebrex), CERE-110, cerebrolysin, CHF 5074, choline alfoscerate, cholinergic antagonist, cholinesterase inhibitor, cilostazol, circadin, citalopram, CMS121, CNP520, COR388, CORT108297, CPC-201, crenezumab, CT1812, CX516, CY6463, DAOI-A, DAOI-B, DAOI-C, dapagliflozin, daratumumab injection, dasatinib, DCB-AD1, deferiprone, delta-9-tetrahydrocannabinol, dex-methylphenidate, dextroamphetamine, DHP1401, dimebon, divalproex, divalproex ER, dl-alpha-tocopherol, docosahexaenoic acid, donanemab, donepezil, donepezil/memantine, donepezil HCL, dronabinol, DSPE-DOTA-Gd liposomal onjection, E-400 clear, E-600, E-Gems, E2020, E2609, E2814, ebicomb, ebixa, EGb761, EHT 0202 etazolate, elenbecestat (E2609), ELND005, elontril, empagliflozin, emtriva capsule, ENA713, E-Pherol, epigallocatechin-gallate, ergoloid mesylates, escitalopram, escitalopram oxalate, estrogen, etanercept, ethosuximide, EVP-0962, EVP-6124, EX039, exelon, exendin-4 SC, ExPlas, filgrastim, FK962, florbetaben (BAY94-9172), flos gossypii flavonoids, flutemetamol, formoterol A, gabapentin enacarbil, galantamine, galantamine hydrobromide, galsya XL, gantenerumab, gastro-retentive zinc cysteine, gatalin XL, gonadorelin acetate, GRF6019, GSK239512, guanfacine, GV-971, GV1001, haloperidol, HB-adMSCs, HF0220, human, esenchymal stem cells and lactated riunger's solution, huperzine A, hydergine, hydralazine hydrochloride, hydrocodone/APAP, hydroxypropyl beta cyclodextrin, IBC-Ab002, icosapent ethyl, ID1201, idalopirdine, IGC-AD1, immune globulin intravenous (Human), immunoglobulin, indomethacin, insulin, insulin glulisine, IONIS MAPTRx, isotretinoin, ITI-007, JACI-35.054, JNJ-54861911, JNJ-63733657, KarXT, prazosin, L-arginine, L-serine, ladostigil hemitartrate, lanabecestat, lecanemab, lecozotan SR, lemborexant, lenalidomide, leqembi, leuprolide acetate, levetiracetam, levodopa, liraglutide, lithium, lithium carbonate, LM11A-31-BHS, LMTM, long-chain triglyceride, lornoxicam, losartan, lovostatin, Lu AE58054, Lu AF87908, LX1001, LY450139, LY450139 dihydrate, LY451395, LY2062430, LY2886721, LY3154207, LY3202626, LY3372689, LY3372993, MABT5102A, masitinib, masupirdine, MEDI1814 (antiamyloid beta mAb), medroxyprogesterone, MEM 1003, MEM 3454, memantine, memantine hydrochloride, metformin, methylene blue, methylphenidate, MIB-626, mifepristone, minocycline, mirtazapine, MK-1942, MK-2214, MK-7622, MK0249, MK0677, MK0952, MLC601, MLC901, modafinil, montelukast, montelukast buccal film, MPC-7869, MSDC-0160, MT-4666, MW150, nabilone, namenda XR, namenda, namzaric, NanoLithium® NP03, naproxen, naproxen sodium (Aleve), NE3107, nefiracetam, neflamapimod, nelotanserin, nemdatine, neramexane, NewGam 10% IVIG, NGP 555, NIC5-15, nicotinamide riboside, nilotinib, nilotinib BE, nilvadipine, NI0752, NPO31112, NPT 2042, NS 2330, nuedexta, obicetrapib, octagam 10%, octaplasma, octohydroaminoacridine succinate, olanzapine, omega 3 PUFA, ONO-2506PO, ORM-12741, ORY-2001, paracetamol, PBT2, pepinemab, perindopril, perphenazine, PF-01913539, PF-04360365, PF-04447943, PF-04494700, PF-05212377, PF-06648671, pimavanserin, pimavanserin tartrate, pioglitazone, piracetam, piromelatine, PM012, posiphen, PPI-1019, PQ912, prednisone, probucol, proleukin, PRX-03140, PTI-125, PXT00864, PYM50028, QS-21, quercetin, quetiapine, quetiapine fumarate, raloxifene, ramelteon, ramipril, rapamycin, rasagiline, razadyne, razadyne ER, RB-ADSC, regulatory T cells, REM0046127, reminyl, remternetug, reserpine, resveratrol, rifaximin, rilapladib, riluzole, risperidone, rivastigmine, rivastigmine patch, RO4602522, RO5313534, RO7126209, rofecoxib, rosiglitazone, rosiglitazone XR, rotigotine transdermal patch, RPh201, RVT-101, S-equol, S47445, SAGE-718, SAM-531, SAR110894, sargramostim, SB-742457, SCI-110, seltorexant, semagacestat, semagludtide, semorinemab, senicapoc, sertraline, SGS742, SHR-1707, simufilam, simvastatin, SK-PC-B70M, SLS-005, SNKO1, sodium oligo-mannurarate, sodium oligomannate, sodium oxybate, solanezumab, solifenacin, spironolactone, SR57667B, SRA-333, SSR180711C, ST101, sulbutiamine, SUVN-502, suvorexant, T-817MA, T-817MA-H, T-817MA-L, T2:C100, T3D-959, tacrine, tacrolimus, talsaclidine, tamibarotene, tandospirone citrate, tau mab, TB006, telmisartan, tetrahydrobiopterin, TH9507 human growth hormone releasing hormone, thalidomide, thiethylperazine, tideglusib, tilavonemab, TPI 287 (next generation taxane), tramiprosate, traneurocin, transdermal estradiol, transdermal methylphenidate, trazodone, tricaprilin, troriluzole, TRx0014, TRx0237, tryptophan, TW001, UB-311, UCMSCs, valaciclovir/valacyclovir, valproate, varenicline, venlafaxine, verubecestat, VI-1121, vitamin e, VP4896, VT301, VX-745, xaliproden (SR57746A), xanamem, XPro1595, zagotenemab, zolpidem, zoplicone, or zydena.

In some embodiments, monoclonal or polyclonal antibodies, such as antibodies against beta amyloid, are used to treat a brain disorder. The presently disclosed pharmaceutical compositions of idazoxan may be used in combination with a second therapeutic agent that also is a monoclonal or polyclonal antibody. For example, the presently disclosed pharmaceutical compositions of idazoxan may be used in combination with Aducanumab or Legembi (lecanemab-irmb).

Exemplary Parkinson's treatments: Carbidopa-levodopa, Rytary, Sinemet, Duopa, Inbrija. Dopamine agonists, Pramipexole, Mirapex ER, Rotigotine, Neupro, Apomorphine, Apokyn, monoamine oxidase B (MAO B) inhibitors. Zelpar, Rasagiline, Azilect, selegiline, safinamide, Xadago. Catechol O-methyltransferase (COMT) inhibitors. Entacapone (Comtan) and opicapone (Ongentys), Anticholinergics including benztropine (Cogentin) or trihexyphenidyl, Amantadine (Gocovri), Adenosine receptor antagonists (A2A receptor antagonists), Istradefylline (Nourianz), deep brain stimulation (DBS), MRI-guided focused ultrasound, Nuplazid, pimavanserin, eplivanserin, volinanserin.

Exemplary treatments for other neurological disorders: include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, paliperidone, asenapine, amisulpride, aripiprazole, lurasidone, ziprasidone, lumateperone, perospirone, mosapramine, AMDA (9-Aminomethyl-9,10-dihydroanthracene), methiothepin, xanomeline, buspirone, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, an extended-release form of aripiprazole including Abilify Maintena, 3-(2-(4-(4-Fluorobenzoyl)piperazin-1-yl)ethyl)-5-methyl-5-phenylimidazolidine-2,4-dione, 3-(2-(4-Benzhydrylpiperazin-1-yl)ethyl)-5-methyl-5-phenylimidazolidine-2,4-dione, 3-(3-(4-(2-Fluorophenyl)piperazin-1-yl)propyl)-5-methyl-5-phenylimidazolidine-2,4-dione, 3-(3-(4-(3-Fluorophenyl)piperazin-1-yl)propyl)-5-methyl-5-phenylimidazolidine-2,4-dione, 3-(3-(4-(4-Fluorophenyl)piperazin-1-yl)propyl)-5-methyl-5-phenylimidazolidine-2,4-dione, 3-(3-(4-(4-Fluorobenzoyl)piperazin-1-yl)propyl)-5-methyl-5-phenylimidazolidine-2,4-dione, 3-(2-(4-(4-Fluorobenzoyl)piperazin-1-yl)ethyl)-8-phenyl-1,3-diazaspiro[4.5]decane-2,4-dione, 3-(2-(4-Benzhydrylpiperazin-1-yl)ethyl)-8-phenyl-1,3-diazaspiro[4.5]decane-2,4-dione, 3-(3-(4-(2-Fluorophenyl)piperazin-1-yl)propyl)-8-phenyl-1,3-diazaspiro[4.5]decane-2,4-dione, 3-(3-(4-(3-Fluorophenyl)piperazin-1-yl)propyl)-8-phenyl-1,3-diazaspiro[4.5]decane-2,4-dione, 3-(3-(4-(4-Fluorophenyl)piperazin-1-yl)propyl)-8-phenyl-1,3-diazaspiro[4.5]decane-2,4-dione, and 3-(3-(4-(4-Fluorobenzoyl)piperazin-1-yl)propyl)-8-phenyl-1,3-diazaspiro[4.5]decane-2,4-dione, or a pharmaceutically acceptable salt, solvate, metabolite, deuterated analogue, derivative, prodrug, or combinations thereof

### Computer Based Analysis

Exemplary procedures in accordance with the disclosure described herein can be performed by a cloud-based processing arrangement and/or a computing arrangement (e.g., computer hardware arrangement). Such processing/computing arrangement can be, for example entirely or a part of, or include, but not limited to, a computer/processor that can include, for example one or more microprocessors, and use instructions stored on a computer-accessible medium (e.g., RAM, ROM, hard drive, or other storage device).

For example a computer-accessible medium (e.g., as described herein above, a storage device such as an encrypted cloud file, hard disk, floppy disk, memory stick, CD-ROM, RAM, ROM, etc., or a collection thereof) can be provided (e.g., in communication with the processing arrangement). The computer-accessible medium can contain executable instructions thereon. In addition or alternatively, a storage arrangement can be provided separately from the computer-accessible medium, which can provide the instructions to the processing arrangement so as to configure the processing arrangement to execute certain exemplary procedures, processes, and methods, as described herein above, for example.

Further, the exemplary processing arrangement can be provided with or include an input/output port, which can include, for example a wired network, a wireless network, the internet, an intranet, a data collection probe, a sensor, etc. The exemplary processing arrangement can be in communication with an exemplary display arrangement. The exemplary display arrangement, according to certain exemplary embodiments of the present disclosure, can be a touchscreen configured for inputting information to the processing arrangement in addition to outputting information from the processing arrangement, for example. Further, the exemplary display arrangement and/or a storage arrangement can be used to display and/or store data in a user-accessible format and/or user-readable format.

### Numbered Embodiments

Embodiment 1. A system comprising:
an MRI system configured to generate and acquire a neuromelanin-sensitive MRI scan along with a neuromelanin data series for a voxel or segment located within a region of interest in a subject's brain;
a signal processor configured to process the series of neuromelanin data to produce a processed neuromelanin MRI spectrum; and
a diagnostic processor configured to process the signal processed neuromelanin MRI spectrum, and
extract a level, signal and/or concentration of neuromelanin in the left SN, right SN, left LC, and right LC;
compare the measurement to a pre-determined control level, signal and/or concentration of neuromelanin in the left SN, right SN, left LC, and right LC,
identifying a neurological disorder if the level, signal and/or concentration of neuromelanin in the left SN, right SN, left LC, and right LC deviates from the control by a predetermined amount.

Embodiment 2. A method of treating a neurological disorder comprising:
(i) obtaining a neuromelanin-sensitive Magnetic Resonance Imaging (MRI) scan;
(ii) determining the level, signal and/or concentration of neuromelanin in the left SN, right SN, left LC, and right LC;
(ii) comparing the level, signal and/or concentration of neuromelanin in the left SN, right SN, left LC, and right LC to a pre-determined control level, signal and/or concentration of neuromelanin in the left SN, right SN, left LC, and right LC;
(iii) administering a treatment to the subject if the level, signal and/or concentration of neuromelanin in one or more of the left SN, right SN, left LC, and right LC deviates from the control by a predetermined amount.

Embodiment 3. A method for treating a neurological disorder in a subject in need thereof comprising:
a) performing a neuromelanin-sensitive MRI scan on the subject;
b) acquiring a level, signal and/or concentration of neuromelanin in the left SN, right SN, left LC, and right LC;
c) comparing the level, signal and/or concentration of neuromelanin in the left SN, right SN, left LC, and right LC in a region of interest in the subject's brain with a predetermined control from each region;
d) providing an identification of the neurological disorder based on the comparing of (c);
e) administering to a subject a therapeutic for the treatment of the identified neurological disorder;
f) performing one or more subsequent neuromelanin-sensitive MRI scans on the subject.

Embodiment 4. A method for distinguishing between neurological disorders with similarly presenting symptoms comprising:
(i) performing an examination;
(ii) obtaining a neuromelanin-sensitive Magnetic Resonance Imaging scan at a first time point;
(iv) performing a voxel-based analysis and determining a concentration and/or volume of neuromelanin in the SNc;
(v) performing a segmented based analysis and determining a concentration and/or volume of neuromelanin in the LC;
(vi) comparing the level, concentration and/or volume of neuromelanin in the SNc to a standard value;
(vii) comparing the level, concentration and/or volume of neuromelanin in the LC to a standard value;
(viii) distinguishing between the neurological disorders based on the deviation of the level, concentration and/or volume of neuromelanin SNc and/or LC and a control.

Embodiment 5. The method of embodiment 4, wherein the examination comprises determining a Unified Parkinson's Disease Rating Scale score.

Embodiment 6. A method of diagnosing a subject with a neurological disorder, said method comprising:
(i) measuring a concentration and/or volume of neuromelanin in the SNc using a voxel-based analysis method and measuring a concentration and/or volume of neuromelanin in the LC using a segmented based analysis method;
(ii) comparing the level of neuromelanin in the SNc to a standard control level of neuromelanin in the SNc and comparing the level of neuromelanin in the LC to a standard control level of neuromelanin in the LC,
(iii) providing a diagnosis of the neurological condition if the magnitude or ratio of SNc and LC neuromelanin is lower or higher for each of their respective regions relative to the control.

Embodiment 7. A method of determining if a subject has or is at risk of developing a neurological disorder, the method comprising analyzing one or more neuromelanin-sensitive Magnetic Resonance Imaging scans of the subject's brain region of interest, wherein the analyzing comprises:
receiving imaging information of the brain region of interest; and
determining a neuromelanin level or concentration in the brain region of interest using segmented analysis based on the imaging information;
wherein the determining if a subject has or is at risk of developing the neurological disorder comprises:
(1) if the one or more neuromelanin-sensitive MRI scans has a decreased neuromelanin signal compared to a one or more control level without a neurological disorder then the subject has or is at risk of developing the neurological disorder; or
(2) if the one or more neuromelanin-sensitive MRI scans have a neuromelanin signal within one standard deviation of one or more control level then the subject does not have or is not at risk of developing the neurological disorder.

Embodiment 8. A method for distinguishing between neurological disorders with similarly presenting symptoms comprising:
(i) performing an examination to determine a Unified Parkinson's Disease Rating Scale score;
(ii) obtaining a neuromelanin-sensitive MRI scan;
(iv) performing a voxel-based analysis and determining a level, concentration and/or volume of neuromelanin in the SNc;
(v) performing a segmented based analysis and determining a level, concentration and/or volume of neuromelanin in the LC;
(vi) comparing the level, concentration and/or volume of neuromelanin in the SNc to a standard value;
(vii) comparing the level, concentration and/or volume of neuromelanin in the LC to a standard value;
(viii) identifying the neurological disorder.

Embodiment 9. The system or method according to any one of the previous embodiments, wherein the method further comprises performing a segmented-based algorithm analysis to determine the level, concentration and/or volume of neuromelanin (NM) in the locus coeruleus (LC).

Embodiment 10. The system or method according to any one of the previous embodiments, wherein the method further comprises performing a voxel-based algorithm analysis to determine the level, concentration and/or volume of neuromelanin in the substantia nigra pars compacta (SNc).

Embodiment 11. The system or method according to any one of the previous embodiments, wherein a neurological disorder is identified if the signal and/or concentration of neuromelanin in the SN is greater than or less than a standard value of 10.02±1.48 CNR or 10.28±1.51 CNR.

Embodiment 12. The system or method according to any one of the previous embodiments wherein a neurological disorder is identified if the signal and/or concentration of neuromelanin in the SN is greater than or less than one standard deviation from a control of 10.02±1.48 CNR or 10.28±1.51 CNR.

Embodiment 13. The system or method according to any one of the previous embodiments, wherein a neurological disorder is identified the signal and/or concentration of neuromelanin in the SN is greater than or less than two standard deviations from a control of 10.02±1.48 CNR or 10.28±1.51 CNR.

Embodiment 14. The system or method according to any one of the previous embodiments, wherein a neurological disorder is identified as schizophrenia if the level, signal and/or concentration of neuromelanin in the SN is greater than one standard deviation from a control.

Embodiment 15. The system or method according to any one of the previous embodiments, wherein a neurological disorder is identified as schizophrenia if the level, signal and/or concentration of neuromelanin in the SN is greater than two standard deviations a control.

Embodiment 16. The system or method according to any one of the previous embodiments, wherein a neurological disorder is identified as Parkinson's disease if the level, signal and/or concentration of neuromelanin in the SN is reduced and the magnitude of the reduction is greater than one standard deviation a control.

Embodiment 17. The system or method according to any one of the previous embodiments, wherein a neurological disorder is identified as Parkinson's disease if the level, signal and/or concentration of neuromelanin in the SN is reduced and the magnitude of the reduction is greater than two standard deviations a control.

Embodiment 18. The system or method according to any one of the previous embodiments, wherein a neurological disorder is identified as a parkinsonian disorder including Parkinson's disease, parkinsonian disorders, multiple system atrophy (MSA), progressive supranuclear palsy (PSP) and corticobasal degeneration (CBD) if the level, signal and/or concentration of neuromelanin in the SN is reduced and the magnitude of the reduction is greater than one standard deviation a control.

Embodiment 19. The system or method according to of any one of the previous embodiments, wherein a neurological disorder is identified as a parkinsonian disorder including Parkinson's disease, parkinsonian disorders multiple system atrophy (MSA), progressive supranuclear palsy (PSP) and corticobasal degeneration (CBD) if the level, signal and/or concentration of neuromelanin in the SN is reduced and the magnitude of the reduction is greater than two standard deviations from a control.

Embodiment 20. The system or method according to any one of the previous embodiments, wherein a neurological disorder is identified as Alzheimer's disease if the level, signal and/or concentration of neuromelanin in the LC is reduced and the magnitude of the reduction is greater than one standard deviation from a control.

Embodiment 21. The system or method according to any one of the previous embodiments, wherein a neurological disorder is identified as Alzheimer's disease if the level, signal and/or concentration of neuromelanin in the LC is reduced and the magnitude of the reduction is greater than two standard deviations from a control.

Embodiment 22. The system or method according to any one of the previous embodiments, wherein a neurological disorder is identified as Alzheimer's disease with neuropsychiatric symptoms if the level, signal and/or concentration of neuromelanin in the LC is increased and the magnitude of the reduction is greater than two standard deviations from a control.

Embodiment 23. The system or method according to any one of the previous embodiments, wherein a neurological disorder is identified as Alzheimer's disease with neuropsychiatric symptoms if the level, signal and/or concentration of neuromelanin in the LC is increased and the magnitude of the reduction is greater than one standard deviation from a control.

Embodiment 24. The system or method according to any one of the previous embodiments, wherein a neurological disorder is identified as Parkinson's disease if the level, signal and/or concentration CNR of neuromelanin in the SN is reduced more than one standard deviation from a control and the level, signal and/or concentration CNR of neuromelanin in the LC is also reduced one standard deviation from a control.

Embodiment 25. The system or method according to any one of the previous embodiments, wherein a neurological disorder is identified as multisystem atrophy (Shy Drager syndrome) if the level, signal and/or concentration CNR of neuromelanin in the SN is reduced more than one standard deviation from a control and the level, signal and/or concentration CNR of neuromelanin in the LC is within one standard deviation from a control.

Embodiment 26. The system or method according to any one of the previous embodiments, wherein a neurological disorder is identified as Alzheimer's disease with neuropsychiatric symptoms if the level, signal and/or concentration CNR of neuromelanin in the LC is increased more than one standard deviation from a control and the level, signal and/or concentration CNR of neuromelanin in the SN is within one standard deviation from a control.

Embodiment 27. The system or method according to any one of the previous embodiments, wherein a neurological disorder is identified as schizophrenia if the level, signal and/or concentration CNR of neuromelanin in the SN is increased more than one standard deviation from a control and the level, signal and/or concentration CNR of neuromelanin in the LC is within one standard deviation from a control.

Embodiment 28. The system or method according to any one of the previous embodiments, wherein a neurological disorder is identified as major depressive disorder if the level, signal and/or concentration CNR of neuromelanin in the LC is decreased more than one standard deviation from a control and the level, signal and/or concentration CNR of neuromelanin in the SN is within one standard deviation from a control.

Embodiment 29. The system or method according to any one of the previous embodiments, wherein a neurological disorder is identified as Parkinson's disease if the level, signal and/or concentration and or CNR of neuromelanin in the SN is more than 1 standard deviation decreased from 10.0.

Embodiment 30. The system or method according to any one of the previous embodiments, wherein a neurological disorder is identified as Parkinson's disease if the volume of neuromelanin in the SN is more than 1 standard deviation decreased from 0.52 cm3.

Embodiment 31. The system or method according to any one of the previous embodiments, wherein a neurological disorder is identified as Parkinson's disease if the volume of neuromelanin in the SN is more than 1 standard deviation decreased from 0.6 cm3.

Embodiment 32. The system or method according to any one of the previous embodiments, wherein a neurological disorder is identified as a parkinsonian disorder including Parkinson's disease, parkinsonian disorders, multiple system atrophy (MSA), progressive supranuclear palsy (PSP) and corticobasal degeneration (CBD) if the volume of neuromelanin in the SN is more than 1 standard deviation decreased from 0.52 cm3.

Embodiment 33. The system or method according to any one of the previous embodiments, wherein a neurological disorder is identified a parkinsonian disorder including Parkinson's disease, parkinsonian disorders, multiple system atrophy (MSA), progressive supranuclear palsy (PSP) and corticobasal degeneration (CBD) if the volume of neuromelanin in the SN is more than 2 standard deviation decreased from 0.52 cm3.

Embodiment 34. The system or method according to any one of the previous embodiments, wherein a neurological disorder is identified as Schizophrenia if the level, signal and/or concentration and or CNR of neuromelanin in the SN is more than 1 standard deviation increased from 10.0.

Embodiment 35. The system or method according to any one of the previous embodiments, wherein a neurological disorder is identified as Alzheimer's disease with neuropsychiatric symptoms if the level, signal and/or concentration and or CNR of neuromelanin in the LC is more than 1 standard deviation increased from 24.

Embodiment 36. The system or method according to any one of the previous embodiments, wherein a neurological disorder is identified as major depressive disorder if the level, signal and/or concentration and or CNR of neuromelanin in the LC is more than 1 standard deviation decreased from 24.

Embodiment 37. The system or method according to any one of the previous embodiments, wherein a neurological disorder is identified as Parkinson's disease if the level, signal and/or concentration CNR of neuromelanin in the SN is reduced more than one standard deviation from 10.0 and the level, signal and/or concentration CNR of neuromelanin in the LC is also reduced more than one standard deviation from 24.

Embodiment 38. The system or method according to any one of the previous embodiments, wherein a neurological disorder is identified as multisystem atrophy (Shy Drager syndrome) if the level, signal and/or concentration CNR of neuromelanin in the SN is reduced more than one standard deviation from 10.0 and the level, signal and/or concentration CNR of neuromelanin in the LC is within one standard deviation from 24.

Embodiment 39. The system or method according to any one of the previous embodiments, wherein a neurological disorder is identified as Alzheimer's disease with neuropsychiatric symptoms if the level, signal and/or concentration CNR of neuromelanin in the LC is increased more than one standard deviation from 24 and the level, signal and/or concentration CNR of neuromelanin in the SN is within one standard deviation from 10.0.

Embodiment 40. The system or method according to any one of the previous embodiments, wherein a neurological disorder is identified as schizophrenia if the level, signal and/or concentration CNR of neuromelanin in the SN is increased more than one standard deviation from 10 and the level, signal and/or concentration CNR of neuromelanin in the LC is within one standard deviation from 24.

Embodiment 41. The system or method according to any one of the previous embodiments, wherein a neurological disorder is identified as major depressive disorder if the level, signal and/or concentration CNR of neuromelanin in the LC is decreased more than one standard deviation from 24 and the level, signal and/or concentration CNR of neuromelanin in the SN is within one standard deviation from 10.

Embodiment 42. The system or method according to any one of the previous embodiments, wherein a neurological disorder is identified if the level, signal and/or concentration of neuromelanin in the LC is 24 and the level, signal and/or concentration of neuromelanin in the SN is one or more standard deviations less than 24.

Embodiment 43. The system or method according to any one of the previous embodiments, wherein a neurological disorder if the level, signal and/or concentration of neuromelanin in the LC is 24 and the level, signal and/or concentration of neuromelanin in the SN is one or more standard deviations less than 24.

Embodiment 44. The system or method according to any of the preceding embodiments, wherein a standard level is a level of neuromelanin present at approximately the same levels in a population of subjects, or said standard control is approximately the average level of neuromelanin present in a population of healthy subjects.

Embodiment 45. The system or method according to any one of the preceding embodiments, wherein the system or method further comprises performing a physical examination.

Embodiment 46. The system or method according to any one of the preceding embodiments, wherein the system or method is used in conjunction with a physical and/or mental examination.

Embodiment 47. The system or method according to any one of the preceding embodiments, wherein the system or method is used in conjunction with a physical examination.

Embodiment 48. The system or method according to any one of the preceding embodiments, wherein the system or method is used in conjunction with a mental examination.

Embodiment 49. The system or method according to any one of the preceding embodiments, wherein the system or method further comprises performing a motor physical examination of the subject, and if the subject's motor physical examination is abnormal then a diagnosis of Parkinson's disease is provided.

Embodiment 50. The system or method according to any one of the preceding embodiments, wherein the system or method further comprises performing a motor physical examination of the subject, and if the subject's motor physical examination is abnormal then a treatment for Parkinson's disease is administered.

Embodiment 51. The system or method according to any one of the preceding embodiments, wherein the system or method further comprises performing a mental examination of a subject, and if the subject displays psychosis, then a diagnosis of schizophrenia is provided.

Embodiment 52. The system or method according to any one of the preceding embodiments, wherein the system or method further comprises performing a mental examination of a subject, and if the subject displays psychosis, then a treatment for schizophrenia is administered.

Embodiment 53. The system or method according to any of the preceding embodiments, wherein a neuromelanin gradient phantom is used to measure the level, signal and/or concentration of neuromelanin.

Embodiment 54. The system or method according to any of the preceding embodiments, wherein a neuromelanin phantom concentration gradient is scanned about once per subject, about once an hour, about once a day, about once a week, or about once a month.

Embodiment 55. The system or method according to any of the preceding embodiments, wherein a neuromelanin phantom gradient is scanned daily.

Embodiment 56. The system or method according to any of the preceding embodiments, wherein a neuromelanin phantom gradient is scanned with each subject.

Embodiment 57. The system or method according to any of the preceding embodiments, wherein the subject displays symptoms of Parkinson's disease or dementia with lewy bodies.

Embodiment 58. The system or method according to any of the preceding embodiments, wherein the neuromelanin-sensitive MRI scan distinguishes between Alzheimer's disease and Parkinson's disease and between Alzheimer's disease and dementia with lewy bodies.

Embodiment 59. The system or method according to any of the preceding embodiments, wherein the subject or subject exhibits one or more symptom of Alzheimer's disease.

Embodiment 60. The system or method according to any of the preceding embodiments, wherein a subject is diagnosed with Alzheimer's disease without displaying symptoms.

Embodiment 61. The system or method according to any of the preceding embodiments, further comprising diagnosing the subject as having Alzheimer's disease or as not having Alzheimer's disease; and indicating the diagnosis to a user via a user interface.

Embodiment 62. The system or method according to any of the preceding embodiments, wherein the segmented analysis comprises determining at least one topographical pattern within the brain region of interest.

Embodiment 63. The system or method according to any of the preceding embodiments, wherein the method further comprises a calculation using a value that represents a volume of a neuromelanin segment.

Embodiment 64. The system or method according to any of the preceding embodiments, wherein the method is used with a second imaging method, wherein the second imaging method is selected from the group consisting of positron emission tomography (PET), structural MRI, comprises functional MRI (fMRI), blood oxygen level dependent (BOLD) fMRI, iron sensitive MRI, quantitative susceptibility mapping (QSM), diffusion tensor imaging DTI, and single photon emission computed tomography (SPECT), DaTscan and DaTquant.

Embodiment 65. The system or method according to any of the preceding embodiments, wherein the second imaging method comprises Positron Emission Tomography (PET).

Embodiment 66. The system or method according to any of the preceding embodiments, wherein the second imaging method comprises structural MRI.

Embodiment 67. The system or method according to any of the preceding embodiments, wherein the second imaging method comprises functional MRI (fMRI).

Embodiment 68. The system or method according to any of the preceding embodiments, wherein the second imaging method comprises blood oxygen level dependent (BOLD) fMRI.

Embodiment 69. The system or method according to any of the preceding embodiments, wherein the brain region of interest is the ventral substantia nigra.

Embodiment 70. The system or method according to any of the preceding embodiments, wherein the brain region of interest is the lateral substantia nigra.

Embodiment 71. The system or method according to any of the preceding embodiments, wherein the brain region of interest is the ventrolateral substantia nigra.

Embodiment 72. The system or method according to any of the preceding embodiments, wherein the brain region of interest is the substantia nigra pars compacta (SNpc).

Embodiment 73. The system or method according to any of the preceding embodiments, wherein the brain region of interest is the substantia nigra pars reticulata (SNpr).

Embodiment 74. The system or method according to any of the preceding embodiments, wherein the brain region of interest is the ventral tegmental area (VTA).

Embodiment 75. The system or method according to any of the preceding embodiments, wherein the brain region of interest is the locus coeruleus.

Embodiment 76. The system or method according to any of the preceding embodiments, wherein the second imaging method comprises Positron Emission Tomography (PET).

Embodiment 77. The system or method according to any of the preceding embodiments, wherein the second imaging method comprises structural MRI.

Embodiment 78. The system or method according to any of the preceding embodiments, wherein the second imaging method comprises functional MRI (fMRI).

Embodiment 79. The system or method according to any of the preceding embodiments, wherein the second imaging method comprises blood oxygen level dependent (BOLD) fMRI.

Embodiment 80. The system or method according to any of the preceding embodiments, wherein the analysis focuses on the neuromelanin level, concentration, volume, or pattern within symptom-specific and/or disease-specific voxels in the SNc.

Embodiment 81. The system or method according to any of the preceding embodiments, wherein the analysis focuses on the neuromelanin level, concentration, volume, or pattern within symptom-specific and/or disease-specific segments in the LC.

Embodiment 82. The system or method according to any of the preceding embodiments, wherein the analysis focuses on the neuromelanin level, concentration, volume, or pattern within symptom specific and/or disease-specific voxels in the SNc and the neuromelanin level, concentration, volume, or pattern within disease-specific and/or symptom-specific segments in the LC.

Embodiment 83. The system or method according to any of the preceding embodiments, wherein the analysis focuses on the neuromelanin level, concentration, or volume, within the SNc and the neuromelanin level, concentration, volume, or pattern within disease-specific and/or symptom-specific segments in the LC.

Embodiment 84. The system or method according to any of the preceding embodiments, wherein the analysis focuses on the neuromelanin level, concentration, volume, or pattern within symptom-specific and/or disease-specific voxels in the SNc and the neuromelanin level, concentration, or volume within the LC.

Embodiment 85. The system or method according to any of the preceding embodiments, wherein the neurological condition is selected from schizophrenia, cocaine use disorder, Parkinson's disease, Alzheimer's disease without neuropsychiatric symptoms, neuropsychiatric symptoms of Alzheimer's disease, major depressive disorder, and/or post-traumatic stress disorder.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort can be had to various other embodiments, modifications, and equivalents thereof which can suggest themselves to those skilled in the art without departing from the spirit of the present disclosure and/or scope of the appended claims.

### Example 1. Evaluating Normative Range of Neuromelanin-Sensitive MRI Metrics

To advance neuromelanin-sensitive MRI towards its potential use as a biomarker, we conducted a neuromelanin-sensitive MRI study in a large sample of cognitively normal older adults using standard methods for acquisition and analysis via a fully automated software for processing neuromelanin-sensitive MRI images that was recently developed. While a sample larger than the present one and spanning the entire lifespan will be ultimately necessary to provide granular age adjustment, in the current study, we propose that this sample will be adequate to approximate the normative range of neuromelanin-sensitive MRI metrics in cognitively normal older adults over 53 years old, a clinically relevant age-range given the emergence of common neurological (e.g., neurodegenerative) conditions in older adults. We report measures of neuromelanin-sensitive MRI signal and volume for the SN and LC, as well as their annual change.

### Methods

### Participants

Study participants from the community were enrolled in the Translational Biomarkers of Aging and Dementia (TRIAD) cohort, McGill University Research Centre for Studies in Aging, Montreal, Canada. The participants had a detailed clinical assessment, including the Clinical Dementia Rating Scale (CDR) and Mini-Mental State Examination (MMSE). All participants in the current study were deemed to be cognitively unimpaired, with no objective cognitive impairment and a CDR score of 0. Participants were excluded if they had other inadequately treated conditions, active substance abuse, recent head trauma, or major surgery, or if they had MRI/PET safety contraindication. This study was approved by the Douglas Mental Health University Institute Research Ethics Board and written informed consent was obtained from all participants.

### MRI Acquisition

All neuroimaging data were acquired at the McConnell Brain Imaging Centre of the Montreal Neurological Institute (MNI). MRI was acquired on a 3T Prisma-Fit scanner. neuromelanin-sensitive MRI images were collected via a turbo spin echo (TSE) sequence with the following parameters: repetition time (TR)=600 ms; echo time (TE)=10 ms; flip angle=120°; turbo factor=4; in-plane resolution=0.6875×0.6875 mm²; partial brain coverage overlaying the pons and midbrain with field of view (FoV)=165×220; number of slices=20; slice thickness=1.8 mm; number of averages=7; acquisition time=8.45 min. The image stack was aligned perpendicular to the axis of the brainstem in the region of the pons. Whole-brain, T1-weighted MR images (resolution=1 mm, isotropic) were acquired using an MPRAGE sequence for preprocessing of the neuromelanin-sensitive MRI and PET data. Quality of MRI images was visually inspected for artifacts immediately upon acquisition. In case of suboptimal image quality, scans were repeated, time permitting.

### Preprocessing of Neuromelanin-Sensitive MRI Images

Preprocessing of neuromelanin-sensitive MRI images was performed using a fully-automated, cloud-based software package, NM-101, version 1.0.2 (Terran Biosciences). The initial algorithm steps were necessary for processing of both LC and SN metrics. These included brain extraction of T1-weighted images, spatial normalization of T1-images into standardized MNI space, rigid coregistration of neuromelanin-sensitive MRI images to T1-weighted images, and spatial normalization of neuromelanin-sensitive MRI images into MNI space (resampled at 1 mm, isotropic).

SN metrics were calculated from the spatially normalized images. While in some embodiments, the mask used can be referred to as an SN mask, the mask may, in some implementations, also include the dopaminergic ventral tegmental area (VTA). In some embodiments, the mask may be referred to as a mask for the SN-VTA complex. Intensity normalization determined contrast-to-noise ratio (CNR) for each subject and voxel v as the relative change in neuromelanin-sensitive MRI signal intensity I from a reference region RR of white matter tracts known to have reduced neuromelanin content, the crus cerebri, as: CNRᵥ=(Iᵥ−mode(I_{RR}))/mode(I_{RR}). Images were then spatially smoothed with a 1-mm full-width-at-half-maximum Gaussian kernel. Finally, an overinclusive mask of the SN in MNI space was applied that ensures inclusion of relevant nuclei for all individuals. SN signal was calculated by averaging CNR values for all SN voxels on the left and right sides of this mask. SN volume on the left and right sides was calculated by counting the number of SN voxels in MNI space above a fixed intensity threshold (CNR>7.8%) and multiplying by the volume of one voxel (1 mm³). The intensity threshold was equal to two times the standard deviation calculated from all reference region voxels of all subjects after trimming reference region voxels with extreme intensity values (bottom and top 2.5% of voxels).

LC metrics were calculated from the raw neuromelanin-sensitive MRI images. An LC search space (referred to as the LC search mask, spanning 5 mm in the rostrocaudal axis from MNI space coordinates z=−21 to −26) was created to cover the middle LC (where signal intensity is highest and automated segmentation accuracy is very high, ˜99% accuracy) and exclude the rostral and caudal portions of the structure. This LC search mask was then warped to neuromelanin-sensitive MRI space using the inverse transformations generated in the spatial normalization and rigid coregistration steps. This warped LC search mask defined a search space wherein to find the LC for each participant. A cluster-forming algorithm was used to segment the LC within this space, defined as the 3 adjacent voxels with the highest mean signal (total area=1.42 mm², consistent with an LC cross-sectional diameter of 1.34 mm). This was repeated on each side (left and right) and axial slice. To minimize partial volume effects, only the brightest of these voxels (peak intensity voxel) was retained for calculation of LC signal on each side and slice (on the assumption that the peak intensity voxel had the highest fraction of LC tissue. CNR for each voxel v in a given axial slice was then calculated as the relative difference in neuromelanin-sensitive MRI signal intensity I from the portion of a reference region in the same axial slice RR as: CNRᵥ=(Iᵥ−mode(I_{RR}))/mode(I_{RR}). We used a reference region with low neuromelanin concentration, the central pons. The central pons mask was transformed from MNI space and transformed to neuromelanin-sensitive MRI space for each participant. LC signal was calculated by averaging CNR values from the all peak intensity LC voxels on the left and right sides.

To calculate a measure of LC volume, the number of voxels above a fixed intensity threshold were counted on one side for every axial slice, then divided by the number of axial slices to estimate average LC voxels per slice, and then multiplied by the area per voxel (0.47 mm²). This area measure was multiplied by a fixed LC length (equal to the length of the LC search mask in MNI space, 5 mm). Variability in the measure of LC volume could be traced to variability in LC area, with rostrocaudal length fixed due to the inability of our method to precisely measure this dimension. This was repeated on both the left and right sides. This method for the measure of LC volume calculation was designed to capture volume loss in neurodegenerative illness (not necessarily to best assay naturally variability in LC volume in healthy individuals), consistent with the intent of the NM-101 software. The intensity threshold was set as the maximum value where no cognitively normal participants had floor values (0 voxels) for LC volume. Many participants had ceiling values for LC volume using these thresholds. Due to left-right asymmetry in the neuromelanin-sensitive MRI images, different thresholds were applied on the left (CNR=13.5%) and right (CNR=9.7%) sides.

### Statistical Analysis

All neuromelanin-sensitive MRI metrics generated by NM-101 software were analyzed using Matlab software. Normality was assessed using Lilliefors test (alpha=0.05). Linear regression was used to relate baseline and change neuromelanin-sensitive MRI metrics to age. Annual change metrics for all neuromelanin-sensitive MRI metrics were calculated by subtracting neuromelanin-sensitive MRI metrics at baseline from the same metrics at follow-up, 9-16 months later. 1-sample t-tests were conducted to determine if these change metrics significantly differed from 0.

### Results

### Section 1. Normative Metrics: Baseline

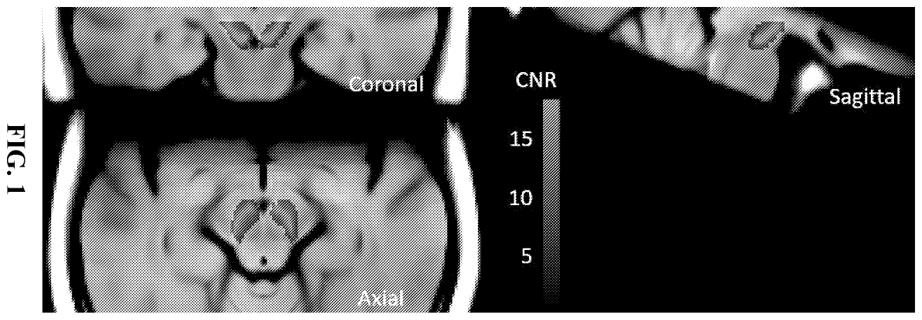

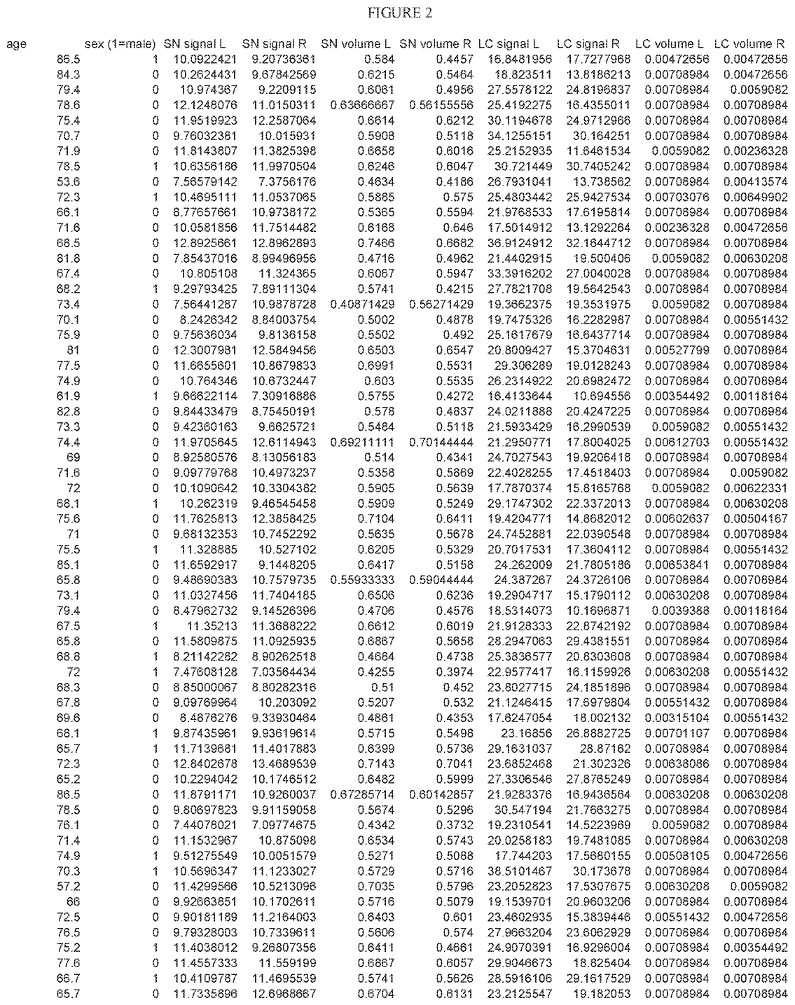

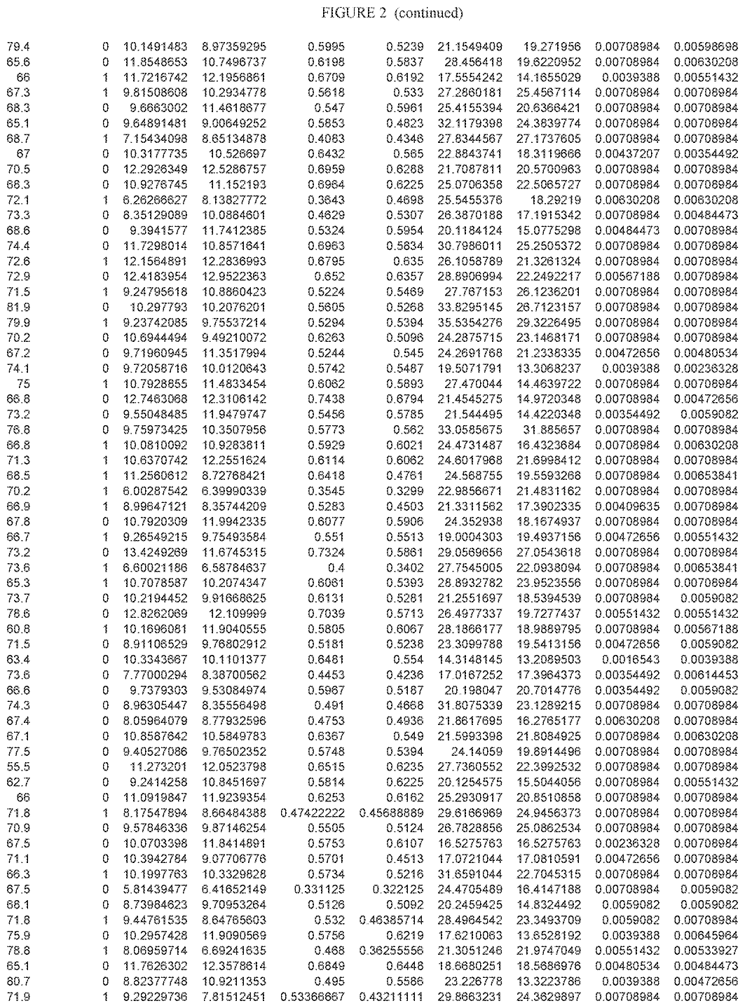

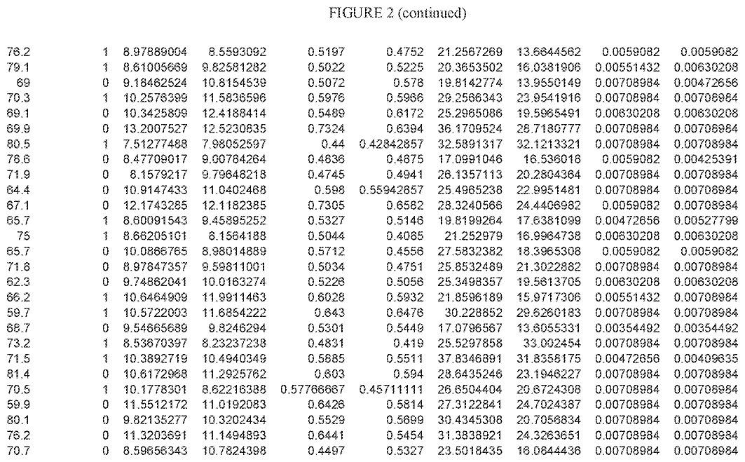

The sample included 152 adults aged between 53 and 86 years at baseline. Mean age was 71.2±5.9 years, mean years of education was 15.3±35, mean score on the mini-mental exam was 29.2±1.4, 50 participants were male (32.9%), and 110 participants were Caucasian (86.6%). Baseline SN signal had CNR values of 10.02% (left SN) and 10.28% (right SN) and baseline LC signal was 24.71% (left) and 20.42% (right; see Table 1 for all signal and volume measures). All signal measures were normally distributed at baseline (Lilliefors test, alpha>0.05). This was not the case for all volume measures (consistent with our decision to set volume thresholds where many healthy individuals would be at-or-near the ceiling, this was particularly true of LC volume metrics). Refer to FIG. 2 (Table) for values of all baseline summary metrics for all participants arising from voxel-level values within the SN for all participants. FIG. 1 shows an SN map illustrating voxelwise values for baseline SN signal.

Given the decision to determine normative neuromelanin-sensitive MRI metrics from a relatively broad age range, we checked whether there was significant (P<0.05) variation in these metrics over this range. We found that there was no statistically significant relation of neuromelanin-sensitive MRI signal or volume in the SN or LC to age (p values ranged from 0.14 to 0.99, linear regression analysis). This indicated an acceptability of the broad range of values and its consistency with evidence that the age-related change in neuromelanin-sensitive MRI metrics plateaus in later life.

### Section 2. Normative Metrics: Annual Change

Follow up images after 1 year (9-16 months) were available for n=41 participants (n=40 for LC). Mean change in neuromelanin-sensitive MRI metrics over the year were all negative (decreases over time) for SN metrics and tended to be positive (increases) for LC metrics (Table 1). There was a large amount of variability across subjects in all of these metrics with some subjects showing increases and some decreases over time. Similar to observations at baseline, annual change in signal metrics was normally distributed but change in volume measures was not. Only SN volume on the left side showed significant annual change across subjects (Table 1, 1-sample t-tests).

TABLE 1
Summary neuromelanin-sensitive MRI metrics at baseline and change over 1 year

| | | SN | | | LC | | |
|---|---|---|---|---|---|---|---|
| | | | | T stat | | | T stat |
| | | | Annual | (annual | | Annual | (annual |
| | | Baseline | change | change) | Baseline | change | change) |
| Signal | Left | 10.02 ± 1.48 | −0.39 ± 1.32 | −1.89 | 24.71 ± 4.88 | 0.67 ± 3.29 | 1.28 |
| (CNR) | Right | 10.28 ± 1.51 | −0.30 ± 1.18 | −1.61 | 20.42 ± 4.95 | −0.62 ± 2.92 | −1.35 |
| Volume | Left | 576.4 ± 83.0 | −30.44 ± 75.9 | −2.57* | 6.31 ± 1.22 | 0.27 ± 0.92 | 1.83 |
| (mm³) | Right | 540 ± 74.9 | −17.21 ± 64.2 | −1.72 | 6.30 ± 1.20 | 0.30 ± 1.37 | 1.41 |

*p = 0.014

Next we determined if age significantly affected annual change metrics. We found this was not the case (p values ranged from 0.16 to 0.96, linear regression analysis) suggesting that, similar to baseline metrics, collapsing older adults across this wide age range may be acceptable when considering annual change metrics.

### Discussion

Normative values for baseline neuromelanin-sensitive MRI signal and volume in the LC and SN and their change over time that were obtained using a fully automated algorithm are reported. Annual change for neuromelanin-sensitive MRI summary metrics where largely non-significant but tended to decrease for SN metrics and increase for LC metrics.

These normative values from cognitively normal older individuals are useful for neuromelanin-sensitive MRI achieving potential as a biomarker to support clinical decision-making. For instance, neuromelanin-sensitive MRI can assist with diagnosis or treatment monitoring by flagging individuals with low neuromelanin-sensitive MRI signal or with marked signal loss over time relative to the norms from healthy individuals. When possible, these normative values would be adjusted in a scanner-specific manner (for instance using a harmonization method and/or system, e.g., ComBat algorithm) to account for differences across scanners that could shift the range of the neuromelanin-sensitive MRI signal, even when parameters and equipment are closely matched between scanners. The subject-level data in Table S1 and Table S2 could assist with this harmonization process.

The sensitivity of the algorithm to detect SN volume changes in the expected direction over a relatively brief time interval provides support for its good performance.

We did not observe statistically significant age-related change in any of our summary neuromelanin-sensitive MRI metrics over the age range employed here. This indicated that these normative values could be used as a reference for adults aged between 53 and 86 years old.

It is also important to note that these normative values are not only limited to a specific population (older adults) but also by the neuromelanin-sensitive MRI acquisition protocol. While they should be robust to small changes in sequence parameters, assuming the values are harmonized using ComBat or a similar approach, they cannot be applied universally to any neuromelanin-sensitive MRI acquisition. We report values using a TSE sequence and these values cannot be compared to values obtained using the other commonly used sequence for neuromelanin-sensitive MRI, a 2D gradient recalled echo sequence with magnetization transfer pulse (2D-GRE with MT). We selected the TSE sequence here given our objective of providing norms that could be clinically useful and the fact that on some scanner brands a developer-made 2D-GRE sequence is needed, making the latter sequence a step further from clinical implementation.

The results provide normative values of neuromelanin-sensitive MRI signal and volume for the SN and LC in cognitively normal older adults. Such norms represent one of the necessary components in the effort to introduce this promising neuroimaging method into the clinic as a biomarker for neurodegenerative and psychiatric disorders.

### EQUIVALENTS

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

What is claimed is:

1. A system comprising:
an MRI system configured to generate and acquire a neuromelanin-sensitive MRI scan along with a neuromelanin data series for a voxel or segment located within a region of interest in a subject's brain;
a signal processor configured to process the series of neuromelanin data to produce a processed neuromelanin MRI spectrum; and
a diagnostic processor configured to process the signal processed neuromelanin MRI spectrum, and
extract a level, signal and/or concentration of neuromelanin in the left SN, right SN, left LC, and right LC;
compare the measurement to a pre-determined control level, signal and/or concentration of neuromelanin in the left SN, right SN, left LC, and right LC,
identifying a neurological disorder if the level, signal and/or concentration of neuromelanin in the left SN, right SN, left LC, and right LC deviates from the control by a predetermined amount.

2. A method of treating a neurological disorder comprising:
(i) obtaining a neuromelanin-sensitive Magnetic Resonance Imaging (MRI) scan;
(ii) determining the level, signal and/or concentration of neuromelanin in the left SN, right SN, left LC, and right LC;
(ii) comparing the level, signal and/or concentration of neuromelanin in the left SN, right SN, left LC, and right LC to a pre-determined control level, signal and/or concentration of neuromelanin in the left SN, right SN, left LC, and right LC;
(iii) administering a treatment to the subject if the level, signal and/or concentration of neuromelanin in one or more of the left SN, right SN, left LC, and right LC deviates from the control by a predetermined amount.

3. A method for treating a neurological disorder in a subject in need thereof comprising:
g) performing a neuromelanin-sensitive MRI scan on the subject;
h) acquiring a level, signal and/or concentration of neuromelanin in the left SN, right SN, left LC, and right LC;
i) comparing the level, signal and/or concentration of neuromelanin in the left SN, right SN, left LC, and right LC in a region of interest in the subject's brain with a predetermined control from each region;
j) providing an identification of the neurological disorder based on the comparing of (c);
k) administering to a subject a therapeutic for the treatment of the identified neurological disorder;
l) performing one or more subsequent neuromelanin-sensitive MRI scans on the subject.

4. A method for distinguishing between neurological disorders with similarly presenting symptoms comprising:
(i) performing an examination;
(ii) obtaining a neuromelanin-sensitive Magnetic Resonance Imaging scan at a first time point;
(iv) performing a voxel-based analysis and determining a concentration and/or volume of neuromelanin in the SNc;
(v) performing a segmented based analysis and determining a concentration and/or volume of neuromelanin in the LC;
(vi) comparing the level, concentration and/or volume of neuromelanin in the SNc to a standard value;
(vii) comparing the level, concentration and/or volume of neuromelanin in the LC to a standard value;
(viii) distinguishing between the neurological disorders based on the deviation of the level, concentration and/or volume of neuromelanin SNc and/or LC and a control.

5. The method of claim 4, wherein the examination comprises determining a Unified Parkinson's Disease Rating Scale score.

6. A method of diagnosing a subject with a neurological disorder, said method comprising:
(i) measuring a concentration and/or volume of neuromelanin in the SNc using a voxel-based analysis method and measuring a concentration and/or volume of neuromelanin in the LC using a segmented based analysis method;
(ii) comparing the level of neuromelanin in the SNc to a standard control level of neuromelanin in the SNc and comparing the level of neuromelanin in the LC to a standard control level of neuromelanin in the LC,
(iii) providing a diagnosis of the neurological condition if the magnitude or ratio of SNc and LC neuromelanin is lower or higher for each of their respective regions relative to the control.

7. A method of determining if a subject has or is at risk of developing a neurological disorder, the method comprising analyzing one or more neuromelanin-sensitive Magnetic Resonance Imaging scans of the subject's brain region of interest, wherein the analyzing comprises:
receiving imaging information of the brain region of interest; and
determining a neuromelanin level or concentration in the brain region of interest using segmented analysis based on the imaging information;
wherein the determining if a subject has or is at risk of developing the neurological disorder comprises:
(1) if the one or more neuromelanin-sensitive MRI scans has a decreased neuromelanin signal compared to a one or more control level without a neurological disorder then the subject has or is at risk of developing the neurological disorder; or
(2) if the one or more neuromelanin-sensitive MRI scans have a neuromelanin signal within one standard deviation of one or more control level then the subject does not have or is not at risk of developing the neurological disorder.

8. A method for distinguishing between neurological disorders with similarly presenting symptoms comprising:
(i) performing an examination to determine a Unified Parkinson's Disease Rating Scale score;
(ii) obtaining a neuromelanin-sensitive MRI scan;
(iv) performing a voxel-based analysis and determining a level, concentration and/or volume of neuromelanin in the SNc;
(v) performing a segmented based analysis and determining a level, concentration and/or volume of neuromelanin in the LC;
(vi) comparing the level, concentration and/or volume of neuromelanin in the SNc to a standard value;
(vii) comparing the level, concentration and/or volume of neuromelanin in the LC to a standard value;
(viii) identifying the neurological disorder.

9. The system or method according to any one of the previous claims, wherein the method further comprises performing a segmented-based algorithm analysis to determine the level, concentration and/or volume of neuromelanin (NM) in the locus coeruleus (LC).

10. The system or method according to any one of the previous claims, wherein the method further comprises performing a voxel-based algorithm analysis to determine the level, concentration and/or volume of neuromelanin in the substantia nigra pars compacta (SNc).

11. The system or method according to any one of the previous claims, wherein a neurological disorder is identified if the signal and/or concentration of neuromelanin in the SN is greater than or less than a standard value of 10.02±1.48 CNR or 10.28±1.51 CNR.

12. The system or method according to any one of the previous claims wherein a neurological disorder is identified if the signal and/or concentration of neuromelanin in the SN is greater than or less than one standard deviation from a control of 10.02±1.48 CNR or 10.28±1.51 CNR.

13. The system or method according to any one of the previous claims, wherein a neurological disorder is identified the signal and/or concentration of neuromelanin in the SN is greater than or less than two standard deviations from a control of 10.02±1.48 CNR or 10.28±1.51 CNR.

14. The system or method according to any one of the previous claims, wherein a neurological disorder is identified as schizophrenia if the level, signal and/or concentration of neuromelanin in the SN is greater than one standard deviation from a control.

15. The system or method according to any one of the previous claims, wherein a neurological disorder is identified as schizophrenia if the level, signal and/or concentration of neuromelanin in the SN is greater than two standard deviations a control.

16. The system or method according to any one of the previous claims, wherein a neurological disorder is identified as Parkinson's disease if the level, signal and/or concentration of neuromelanin in the SN is reduced and the magnitude of the reduction is greater than one standard deviation a control.

17. The system or method according to any one of the previous claims, wherein a neurological disorder is identified as Parkinson's disease if the level, signal and/or concentration of neuromelanin in the SN is reduced and the magnitude of the reduction is greater than two standard deviations a control.

18. The system or method according to any one of the previous claims, wherein a neurological disorder is identified as a parkinsonian disorder including Parkinson's disease, parkinsonian disorders, multiple system atrophy (MSA), progressive supranuclear palsy (PSP) and corticobasal degeneration (CBD) if the level, signal and/or concentration of neuromelanin in the SN is reduced and the magnitude of the reduction is greater than one standard deviation a control.

19. The system or method according to of any one of the previous claims, wherein a neurological disorder is identified as a parkinsonian disorder including Parkinson's disease, parkinsonian disorders multiple system atrophy (MSA), progressive supranuclear palsy (PSP) and corticobasal degeneration (CBD) if the level, signal and/or concentration of neuromelanin in the SN is reduced and the magnitude of the reduction is greater than two standard deviations from a control.

20. The system or method according to any one of the previous claims, wherein a neurological disorder is identified as Alzheimer's disease if the level, signal and/or concentration of neuromelanin in the LC is reduced and the magnitude of the reduction is greater than one standard deviation from a control.

21. The system or method according to any one of the previous claims, wherein a neurological disorder is identified as Alzheimer's disease if the level, signal and/or concentration of neuromelanin in the LC is reduced and the magnitude of the reduction is greater than two standard deviations from a control.

22. The system or method according to any one of the previous claims, wherein a neurological disorder is identified as Alzheimer's disease with neuropsychiatric symptoms if the level, signal and/or concentration of neuromelanin in the LC is increased and the magnitude of the reduction is greater than two standard deviations from a control.

23. The system or method according to any one of the previous claims, wherein a neurological disorder is identified as Alzheimer's disease with neuropsychiatric symptoms if the level, signal and/or concentration of neuromelanin in the LC is increased and the magnitude of the reduction is greater than one standard deviation from a control.

24. The system or method according to any one of the previous claims, wherein a neurological disorder is identified as Parkinson's disease if the level, signal and/or concentration CNR of neuromelanin in the SN is reduced more than one standard deviation from a control and the level, signal and/or concentration CNR of neuromelanin in the LC is also reduced one standard deviation from a control.

25. The system or method according to any one of the previous claims, wherein a neurological disorder is identified as multisystem atrophy (Shy Drager syndrome) if the level, signal and/or concentration CNR of neuromelanin in the SN is reduced more than one standard deviation from a control and the level, signal and/or concentration CNR of neuromelanin in the LC is within one standard deviation from a control.

26. The system or method according to any one of the previous claims, wherein a neurological disorder is identified as Alzheimer's disease with neuropsychiatric symptoms if the level, signal and/or concentration CNR of neuromelanin in the LC is increased more than one standard deviation from a control and the level, signal and/or concentration CNR of neuromelanin in the SN is within one standard deviation from a control.

27. The system or method according to any one of the previous claims, wherein a neurological disorder is identified as schizophrenia if the level, signal and/or concentration CNR of neuromelanin in the SN is increased more than one standard deviation from a control and the level, signal and/or concentration CNR of neuromelanin in the LC is within one standard deviation from a control.

28. The system or method according to any one of the previous claims, wherein a neurological disorder is identified as major depressive disorder if the level, signal and/or concentration CNR of neuromelanin in the LC is decreased more than one standard deviation from a control and the level, signal and/or concentration CNR of neuromelanin in the SN is within one standard deviation from a control.

29. The system or method according to any one of the previous claims, wherein a neurological disorder is identified as Parkinson's disease if the level, signal and/or concentration and or CNR of neuromelanin in the SN is more than 1 standard deviation decreased from 10.0.

30. The system or method according to any one of the previous claims, wherein a neurological disorder is identified as Parkinson's disease if the volume of neuromelanin in the SN is more than 1 standard deviation decreased from 0.52 cm³.

31. The system or method according to any one of the previous claims, wherein a neurological disorder is identified as Parkinson's disease if the volume of neuromelanin in the SN is more than 1 standard deviation decreased from 0.6 cm³.

32. The system or method according to any one of the previous claims, wherein a neurological disorder is identified as a parkinsonian disorder including Parkinson's disease, parkinsonian disorders, multiple system atrophy (MSA), progressive supranuclear palsy (PSP) and corticobasal degeneration (CBD) if the volume of neuromelanin in the SN is more than 1 standard deviation decreased from 0.52 cm³.

33. The system or method according to any one of the previous claims, wherein a neurological disorder is identified a parkinsonian disorder including Parkinson's disease, parkinsonian disorders, multiple system atrophy (MSA), progressive supranuclear palsy (PSP) and corticobasal degeneration (CBD) if the volume of neuromelanin in the SN is more than 2 standard deviation decreased from 0.52 cm³.

34. The system or method according to any one of the previous claims, wherein a neurological disorder is identified as Schizophrenia if the level, signal and/or concentration and or CNR of neuromelanin in the SN is more than 1 standard deviation increased from 10.0.

35. The system or method according to any one of the previous claims, wherein a neurological disorder is identified as Alzheimer's disease with neuropsychiatric symptoms if the level, signal and/or concentration and or CNR of neuromelanin in the LC is more than 1 standard deviation increased from 24.

36. The system or method according to any one of the previous claims, wherein a neurological disorder is identified as major depressive disorder if the level, signal and/or concentration and or CNR of neuromelanin in the LC is more than 1 standard deviation decreased from 24.

37. The system or method according to any one of the previous claims, wherein a neurological disorder is identified as Parkinson's disease if the level, signal and/or concentration CNR of neuromelanin in the SN is reduced more than one standard deviation from 10.0 and the level, signal and/or concentration CNR of neuromelanin in the LC is also reduced more than one standard deviation from 24.

38. The system or method according to any one of the previous claims, wherein a neurological disorder is identified as multisystem atrophy (Shy Drager syndrome) if the level, signal and/or concentration CNR of neuromelanin in the SN is reduced more than one standard deviation from 10.0 and the level, signal and/or concentration CNR of neuromelanin in the LC is within one standard deviation from 24.

39. The system or method according to any one of the previous claims, wherein a neurological disorder is identified as Alzheimer's disease with neuropsychiatric symptoms if the level, signal and/or concentration CNR of neuromelanin in the LC is increased more than one standard deviation from 24 and the level, signal and/or concentration CNR of neuromelanin in the SN is within one standard deviation from 10.0.

40. The system or method according to any one of the previous claims, wherein a neurological disorder is identified as schizophrenia if the level, signal and/or concentration CNR of neuromelanin in the SN is increased more than one standard deviation from 10 and the level, signal and/or concentration CNR of neuromelanin in the LC is within one standard deviation from 24.

41. The system or method according to any one of the previous claims, wherein a neurological disorder is identified as major depressive disorder if the level, signal and/or concentration CNR of neuromelanin in the LC is decreased more than one standard deviation from 24 and the level, signal and/or concentration CNR of neuromelanin in the SN is within one standard deviation from 10.

42. The system or method according to any one of the previous claims, wherein a neurological disorder is identified if the level, signal and/or concentration of neuromelanin in the LC is 24 and the level, signal and/or concentration of neuromelanin in the SN is one or more standard deviations less than 24.

43. The system or method according to any one of the previous claims, wherein a neurological disorder if the level, signal and/or concentration of neuromelanin in the LC is 24 and the level, signal and/or concentration of neuromelanin in the SN is one or more standard deviations less than 24.

44. The system or method according to any of the preceding claims, wherein a standard level is a level of neuromelanin present at approximately the same levels in a population of subjects, or said standard control is approximately the average level of neuromelanin present in a population of healthy subjects.

45. The system or method according to any one of the preceding claims, wherein the system or method further comprises performing a physical examination.

46. The system or method according to any one of the preceding claims, wherein the system or method is used in conjunction with a physical and/or mental examination.

47. The system or method according to any one of the preceding claims, wherein the system or method is used in conjunction with a physical examination.

48. The system or method according to any one of the preceding claims, wherein the system or method is used in conjunction with a mental examination.

49. The system or method according to any one of the preceding claims, wherein the system or method further comprises performing a motor physical examination of the subject, and if the subject's motor physical examination is abnormal then a diagnosis of Parkinson's disease is provided.

50. The system or method according to any one of the preceding claims, wherein the system or method further comprises performing a motor physical examination of the subject, and if the subject's motor physical examination is abnormal then a treatment for Parkinson's disease is administered.

51. The system or method according to any one of the preceding claims, wherein the system or method further comprises performing a mental examination of a subject, and if the subject displays psychosis, then a diagnosis of schizophrenia is provided.

52. The system or method according to any one of the preceding claims, wherein the system or method further comprises performing a mental examination of a subject, and if the subject displays psychosis, then a treatment for schizophrenia is administered.

53. The system or method according to any of the preceding claims, wherein a neuromelanin gradient phantom is used to measure the level, signal and/or concentration of neuromelanin.

54. The system or method according to any of the preceding claims, wherein a neuromelanin phantom concentration gradient is scanned about once per subject, about once an hour, about once a day, about once a week, or about once a month.

55. The system or method according to any of the preceding claims, wherein a neuromelanin phantom gradient is scanned daily.

56. The system or method according to any of the preceding claims, wherein a neuromelanin phantom gradient is scanned with each subject.

57. The system or method according to any of the preceding claims, wherein the subject displays symptoms of Parkinson's disease or dementia with lewy bodies.

58. The system or method according to any of the preceding claims, wherein the neuromelanin-sensitive MRI scan distinguishes between Alzheimer's disease and Parkinson's disease and between Alzheimer's disease and dementia with lewy bodies.

59. The system or method according to any of the preceding claims, wherein the subject or subject exhibits one or more symptom of Alzheimer's disease.

60. The system or method according to any of the preceding claims, wherein a subject is diagnosed with Alzheimer's disease without displaying symptoms.

61. The system or method according to any of the preceding claims, further comprising diagnosing the subject as having Alzheimer's disease or as not having Alzheimer's disease; and indicating the diagnosis to a user via a user interface.

62. The system or method according to any of the preceding claims, wherein the segmented analysis comprises determining at least one topographical pattern within the brain region of interest.

63. The system or method according to any of the preceding claims, wherein the method further comprises a calculation using a value that represents a volume of a neuromelanin segment.

64. The system or method according to any of the preceding claims, wherein the method is used with a second imaging method, wherein the second imaging method is selected from the group consisting of positron emission tomography (PET), structural MRI, comprises functional MRI (fMRI), blood oxygen level dependent (BOLD) fMRI, iron sensitive MRI, quantitative susceptibility mapping (QSM), diffusion tensor imaging DTI, and single photon emission computed tomography (SPECT), DaTscan and DaTquant.

65. The system or method according to any of the preceding claims, wherein the second imaging method comprises Positron Emission Tomography (PET).

66. The system or method according to any of the preceding claims, wherein the second imaging method comprises structural MRI.

67. The system or method according to any of the preceding claims, wherein the second imaging method comprises functional MRI (fMRI).

68. The system or method according to any of the preceding claims, wherein the second imaging method comprises blood oxygen level dependent (BOLD) fMRI.

69. The system or method according to any of the preceding claims, wherein the brain region of interest is the ventral substantia nigra.

70. The system or method according to any of the preceding claims, wherein the brain region of interest is the lateral substantia nigra.

71. The system or method according to any of the preceding claims, wherein the brain region of interest is the ventrolateral substantia nigra.

72. The system or method according to any of the preceding claims, wherein the brain region of interest is the substantia nigra pars compacta (SNpc).

73. The system or method according to any of the preceding claims, wherein the brain region of interest is the substantia nigra pars reticulata (SNpr).

74. The system or method according to any of the preceding claims, wherein the brain region of interest is the ventral tegmental area (VTA).

75. The system or method according to any of the preceding claims, wherein the brain region of interest is the locus coeruleus.

76. The system or method according to any of the preceding claims, wherein the second imaging method comprises Positron Emission Tomography (PET).

77. The system or method according to any of the preceding claims, wherein the second imaging method comprises structural MRI.

78. The system or method according to any of the preceding claims, wherein the second imaging method comprises functional MRI (fMRI).

79. The system or method according to any of the preceding claims, wherein the second imaging method comprises blood oxygen level dependent (BOLD) fMRI.

80. The system or method according to any of the preceding claims, wherein the analysis focuses on the neuromelanin level, concentration, volume, or pattern within symptom-specific and/or disease-specific voxels in the SNc.

81. The system or method according to any of the preceding claims, wherein the analysis focuses on the neuromelanin level, concentration, volume, or pattern within symptom-specific and/or disease-specific segments in the LC.

82. The system or method according to any of the preceding claims, wherein the analysis focuses on the neuromelanin level, concentration, volume, or pattern within symptom specific and/or disease-specific voxels in the SNc and the neuromelanin level, concentration, volume, or pattern within disease-specific and/or symptom-specific segments in the LC.

83. The system or method according to any of the preceding claims, wherein the analysis focuses on the neuromelanin level, concentration, or volume, within the SNc and the neuromelanin level, concentration, volume, or pattern within disease-specific and/or symptom-specific segments in the LC.

84. The system or method according to any of the preceding claims, wherein the analysis focuses on the neuromelanin level, concentration, volume, or pattern within symptom-specific and/or disease-specific voxels in the SNc and the neuromelanin level, concentration, or volume within the LC.

85. The system or method according to any of the preceding claims, wherein the neurological condition is selected from schizophrenia, cocaine use disorder, Parkinson's disease, Alzheimer's disease without neuropsychiatric symptoms, neuropsychiatric symptoms of Alzheimer's disease, major depressive disorder, and/or post-traumatic stress disorder.
